(19) United States
(12) Patent Application Publication
(10) Pub. No.: US 2025/0093289 A1
(43) Pub. Date: Mar. 20, 2025

(54) Method and apparatus for determining properties of containers made of paper or cardboard

(71) Applicant: KRONES AG, Neutraubling (DE)

(72) Inventors: Anton Niedermeier, Offenstetten (DE); Stefan Piana, Koefering (DE)

(21) Appl. No.: 18/888,494

(22) Filed: Sep. 18, 2024

(30) Foreign Application Priority Data
Sep. 18, 2023 (DE) 10 2023 125 199.6

(51) Int. Cl. G01N 27/22; G01B 7/06; G01N 33/34

(52) U.S. Cl. CPC G01N 27/223; G01B 7/08; G01N 33/343

(57) ABSTRACT

A method for determining container properties, wherein a container to be examined is transported along a predetermined transport path, and this container has a bottom region, a main body which serves to receive a filling material, and an mouth region, and wherein this container is formed at least in part of a material containing paper or cardboard. At least one wall of the container is arranged between two electrodes, and an electrical voltage or an alternating electrical field is applied to the electrodes, and at least one electrical parameter is determined which is characteristic of an electrical field which is built up between electrodes, and from this electrical parameter, at least one parameter characteristic of the container is determined.

### BACKGROUND OF THE INVENTION

The present invention relates to a method and to an apparatus for determining container properties. Such methods and apparatuses have long been known from the prior art. For example, it is known that during the production of plastic containers, properties thereof are determined-such as a wall thickness of the container or the like. These properties are important in order to draw conclusions about the quality of the manufactured container, for example whether the determined wall thickness is within a target range.

These methods usually use optical measuring devices, for example the container is illuminated with light, and a transmission is determined from which the container wall thickness can be deduced.

Recently, there has been an increasing effort to produce environmentally friendly alternatives for containers. One approach seeks to produce containers from pulp, in particular paper or cardboard pulp. The first tests are promising. However, such containers have the disadvantage that they usually cannot be inspected with visible light because they do not transmit it.

In the prior art, beverage containers in the form of paper-mâché containers, pulp containers, fiber-molded containers and the like are already known, and are usually produced from an aqueous measure in several process steps. After sufficient drying, this type of container can be filled and closed. However, it is further known that pulp shrinks during drying. In this way, a check of dimensional accuracy, for example, can only be carried out after sufficient drying.

A further problem with such containers is that they often still contain residual moisture. The current prior art makes it possible to determine residual moisture during cardboard quality control, for example using a hygrometer. This measurement is made by contact with a sword sensor. A sword sensor must be in contact with the material until a sufficiently stable measurement value is obtained.

However, these methods are not suitable for the examination of the containers relevant here. In particular, contact measurement would have a high impact on the performance of corresponding machines.

As mentioned, after the production of such pulp containers and therefore usually before further processing (filling, closing and the like), there is still residual moisture in the container wall. This should not exceed a defined value so that the container can be handled reasonably in the facility, during storage and the like (in particular with regard to stability, abrasion resistance, protection against mold and the like). As mentioned above, there are various established methods or ideas for determining residual moisture, such as weighing, radiometric absorption measurement and the like. However, these methods are either slow, complex or expensive, or cannot accurately distinguish between differences in wall thickness and different residual moisture contents.

The present invention is therefore based on the object of providing a determination method and also a determination apparatus which can be used in particular for the containers described here which are made of pulp.

### SUMMARY OF THE INVENTION

In a first method according to the invention for determining container properties, a container to be examined is transported along a predetermined transport path, and this container has a bottom region, a main body which serves to receive the filling material, and a mouth region. Furthermore, this container is at least partially and preferably substantially and particularly preferably completely made of a material containing paper or cardboard and/or is made of such a material.

According to the invention, at least one wall of the container is arranged between two electrodes, and an electrical voltage or an alternating electrical field is applied to the electrodes, and at least one electrical parameter is determined which is characteristic of an electrical field which is built up between these electrodes, and from this electrical parameter, at least one parameter which is characteristic of the container is determined.

In the inventive procedure described herein, the container itself or a wall or a portion of the wall is examined in order to derive a characteristic parameter therefrom, for example a residual moisture content or a wall thickness.

Preferably, the material of the container has a specified liquid—and in particular, water—content.

In particular, said water content and/or the residual moisture distinguishes the container from containers known from the prior art, which are made in particular of glass or plastic. The applicant has therefore developed solutions which are in particular suitable for use with water-containing materials and/or which are suitable for use with paper and/or cardboard materials.

In a preferred embodiment, the container is made of paper and/or cardboard and preferably of a paper or cardboard-containing pulp.

Preferably, a mouth of the container has a smaller cross-section than that of the main body of the container.

In addition, a further approach to the examination was also identified-namely, the above-mentioned possibility of measuring an electric field, and therefore a capacitive measurement.

In a preferred method, one of the two electrodes is located outside the container during the determination of the electrical parameter, and the other electrode is either located outside the container in such a way that the container (in particular at least in part) and in particular its main body (in particular at least in part) is arranged between the two electrodes, or the other electrode is located in part inside the container in such a way that at least one wall portion of the container is located between the electrodes.

In one of the two methods described here, at least one electrode, namely the electrode that is located inside the container, is therefore preferably entrained in the transport direction of the containers.

In a further preferred method, an alternating electric field, and in particular a high-frequency alternating electric field, is built up between the electrodes. Preferably, at least one wall forms a dielectric located between the electrodes. Preferably, during the measurement, there is only air and at least one wall of the container between the electrodes.

Capacitive sensors, for example as shortwave radio electronics, are relatively inexpensive, even if the handling of an immersed electrode in the container is necessary. However, it is also possible for measurement to be feasible at much higher frequencies up to the microwave range.

The preferred measurement frequencies range from 2 MHz to 200 GHz, meaning they can cover the entire radio spectrum from shortwave to UKW,

VHF, UHF, microwaves, radar and up to the mm-wave range.

Higher frequencies offer the advantage that they measure more precisely and therefore enable measurements on a small, localized area of the container wall, while lower frequencies capture larger areas.

In a further preferred method, a or the measurement is carried out at or using at least two different (measurement) frequencies:

The first measurement frequency preferably shows a stronger absorption by the hydrocarbon-containing wood fibers of the pulp, and the second frequency preferably shows a stronger absorption by water from the residual moisture in the wall.

This allows the two characteristic parameters of residual moisture and wall thickness to be determined simultaneously.

It may also be possible to carry out an even simpler measurement using only external electrodes. In this way, residual moisture could be assessed not only at one point on a measuring surface, but over the entire circumference of the container. By measuring with electrodes arranged one above the other, as described in detail below, the container can preferably be divided into several vertical zones that can be measured separately.

In a further preferred method, the characteristic parameter is measured during movement of the containers. It should be noted that this preferred variant is possible for all the methods proposed here, in particular also for capacitive measurement.

In a further preferred method, the parameter characteristic of the container is a moisture value and in particular a residual moisture of the container, and/or a wall thickness of at least one wall portion of the container.

In a further preferred method, the container is rotated during the measurement and in particular rotated with respect to its longitudinal direction. In this way, it is possible to measure the entire lateral surface of the container. It is noted that this preferred embodiment is suitable for the capacitive measurement described herein.

In a further preferred method, the above-mentioned electrical parameter is an amplitude and/or a phase of an electric field (in particular built up between the electrodes).

From this parameter, and in particular from both parameters, i.e., both the amplitude and the phase of the electric field, a residual moisture content and possibly also a wall thickness of the container can be determined.

Particularly preferably, a wall thickness of the container is determined from a known or constant residual moisture. It should be noted in this context that the wall thickness is also included in the calculation of the residual moisture. If the residual moisture is known or constant, the wall thickness can also be determined from the measurements.

In a preferred method, an electrode is guided or immersed into the container. This is preferably done via the opening of the container.

Preferably, one or more electrodes are arranged outside the container.

As noted, the amplitude and phase are particularly preferably measured at different high frequencies. Particularly preferably, different geometries (in particular of the electrodes) are also used to carry out simultaneous moisture and wall thickness measurements.

With a preferably implemented rotation of the container (for example by rolling it), it is possible to measure the entire lateral surface.

In the above-mentioned case of a constant, or at least known, residual moisture, a compensation, and therefore a direct wall thickness calculation is possible. In addition, a measurement with external electrodes is also possible, which enables a measurement on a conveyor with unrestricted throughput.

In a further preferred method, the determination of the above-mentioned properties and/or the measurement is carried out without contact. The above-mentioned methods have the advantage that they do not lead to a limitation of production output.

The cited method allows quality control of the produced container. These measured values can also be used to perform subsequent drying of the containers. In this way, closed-loop control of, for example, the material input, a material mixing ratio, a water content, a drying energy, a drying time and the like can also be carried out.

Preferably, the measurements described herein are carried out on an unfilled container. Preferably, the measurements described herein are carried out on an unclosed container.

Preferably, the measurements described herein are carried out on a container that is to be filled for the first time.

The present invention is further directed to an apparatus for determining container properties. The apparatus has a transport device which transports a container to be examined (and preferably a plurality of containers to be examined) along a predetermined transport path, wherein this container has a bottom region, a main body which serves to receive a filling material, and a mouth region, and wherein this container consists at least in part and preferably completely of a material containing paper or cardboard.

According to the invention, the apparatus has at least two electrodes which are arranged such that at least one wall of the container is temporarily arranged between these two electrodes, and wherein an electrical voltage can be applied to the electrodes, and furthermore a measuring device is provided which determines an electrical parameter which is characteristic of an electrical field which can be and/or is built up between the electrodes. Furthermore, an evaluation device is provided which determines at least one parameter which is characteristic of the container from this electrical parameter.

It is therefore also proposed, for the apparatus, that the container is measured without contact. Preferably, the measurement is also inline, i.e. during a production operation.

As mentioned above, capacitive measurements are used to measure the respective parameters.

Preferably, the parameter characteristic of the container is a humidity and/or a moisture content and/or a wall thickness, in particular a wall thickness in a region of the bottom or the main body of the container.

Particularly preferably, the transport device is a (in particular single-track) transport belt or a transport chain. Particularly preferably, the containers are transported along a straight direction and in particular in an upright state.

However, it would also be possible for the transport device to have a rotatable carrier on which a plurality of holding elements for holding the containers are arranged, or for the container to be transported with neck handling, in particular into or out of a cavity.

The electrodes described above preferably extend in a longitudinal direction of the container(s), wherein the container longitudinal direction preferably extends from the mouth to the bottom of the container.

In a preferred embodiment, at least one electrode and in particular an external electrode has a plurality of electrode portions that can be controlled separately or subjected to different frequencies, such that an electric field can preferably be built up between each individual electrode portion of this electrode and the other electrode. In this way, for example, a residual moisture measurement is possible depending on the height position of the container.

In a further preferred embodiment, the apparatus has a rotating device that is suitable and intended for rotating the containers, in particular with respect to their longitudinal direction. It should be noted that this embodiment is applicable both for measurements using electromagnetic radiation and for capacitive measurements.

In one variant, it is possible for this rotation to take place when the measurement is carried out, but it would also be possible to rotate and measure partially, multiple times, along the transport path.

Preferably, the rotating device can, for example, have lateral guide belts which can be applied against the main body of the container and therefore also cause it to rotate.

The present invention is further directed to an arrangement for processing containers, having an apparatus of the type described above, and having a drying device for drying the containers, wherein this drying device is preferably controllable depending on the characteristic parameter of the container, in particular a residual moisture. Drying is preferably carried out with blown-in air which flows through the drying chamber with the container located therein. The parameters to be controlled can be, for example, the temperature, the flow velocity, or vacuum pressure level of the drying air.

Preferably, this drying device is arranged after the apparatus described above. Particularly preferably, the control device allows a control, and in particular a control in the form of a closed loop or a closed control circuit.

Furthermore, the arrangement can comprise several of the apparatuses described above. For example, it is possible to carry out different measurements one after the other along the transport path—for example, a measurement using near-infrared light and a capacitive measurement.

The measurement results can, for example, be used redundantly; it would also be possible to ascertain residual moisture with a first measurement and determine wall thickness with a further measurement. The person skilled in the art will recognize that the apparatuses proposed here can be combined with one another—for example a measurement with X-rays with a measurement with infrared light, or a capacitive measurement with a heat measurement, or further embodiments.

### BRIEF DESCRIPTION OF THE DRAWINGS

Further advantages and embodiments can be seen in the accompanying drawings:

In the drawings:

FIG. 1 shows a schematic illustration of an apparatus;

FIG. 2 shows a schematic illustration of a capacitive measurement; and

FIG. 3 shows a further illustration of a capacitive measurement.

### DETAILED DESCRIPTION OF THE INVENTION

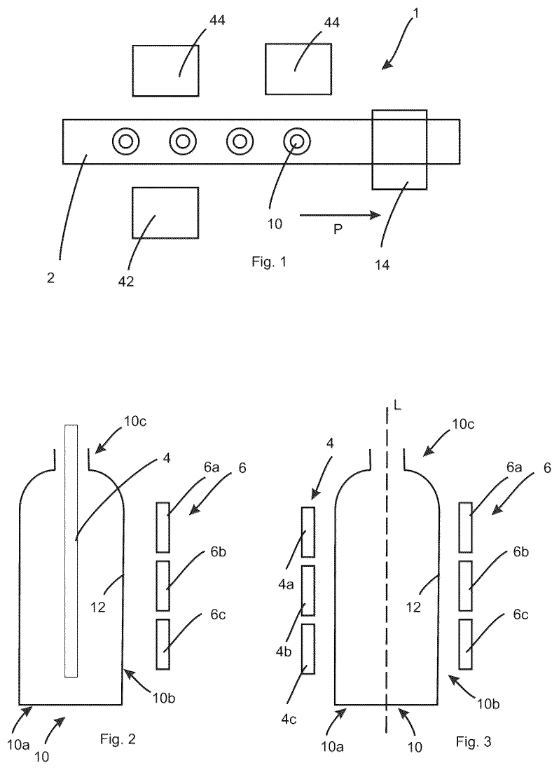

FIG. 1 shows a roughly schematic illustration of an apparatus 1. A transport device 2, such as a transport belt, is provided, which transports containers 10 along a transport path P. Although this figure shows an apparatus which is designed for measurements with electromagnetic radiation, the use of the shown transport belt can also be considered for a capacitive measurement. This apparatus in particular can also be used in a corresponding manner for the capacitive measurement described herein.

The reference sign 42 designates a radiation device which radiates radiation onto the containers 10, and the reference sign 44 designates a sensor device which captures the radiation transmitted or reflected by the containers. In the case of the invention, electrodes could be provided on both sides of the transport belt, which carry out the capacitive measurements described in the context of the present application.

It would also be possible for a first electrode to be arranged next to the transport path, and for a further electrode to be inserted into the containers and/or to move with the containers.

The reference sign 14 denotes a drying device which is used to dry the containers 10. Preferably, a control device is provided for controlling this drying device, which controls this drying process in particular depending on a measured value established by the sensor device, such as in particular a determined residual moisture content of the container.

As mentioned above, the containers are containers that are made of paper or cardboard and in particular a pulp made of paper or cardboard.

FIGS. 2 and 3 show arrangements for a capacitive measurement. In the variant shown in FIG. 2, an electrode 4 is inserted into the container, and the electric field of a second electrode 6 is determined, which here has three individual electrodes 6a, 6b and 6c. From the characteristics of this electric field, in particular the amplitude and the phase, conclusions can be drawn about the residual moisture in the wall 10b of the container.

FIG. 3 shows a further variant, wherein here two electrodes 4 (4a, 4b, 4c) and 6 (6a, 6b, 6c) are each arranged on the outside. The advantage of this measurement is that no electrode has to be moved with the container-which also moves perpendicular to the plane of the figure in this case.

The applicant reserves the right to claim all features disclosed in the application documents as essential to the invention, provided that they are novel over the prior art individually or in combination. It is also pointed out that features which can be advantageous in themselves are also described in the individual figures. The person skilled in the art will immediately recognize that a particular feature described in a figure can be advantageous even without the adoption of further features from this figure. Furthermore, the person skilled in the art will recognize that advantages can also result from a combination of several features shown in individual or in different figures.

1. A method for determining container properties, wherein a container to be examined is transported along a predetermined transport path, and this container has a bottom region, a main body which serves to receive a filling material, and an mouth region, and wherein this container is made at least in part of a material containing paper or cardboard,
wherein
at least one wall of the container is arranged between two electrodes, and an electrical voltage is applied to the electrodes, and at least one electrical parameter is determined which is characteristic of an electrical field which is built up between electrodes, and from this electrical parameter, at least one parameter which is characteristic of the container is determined.

2. The method according to claim 1,
wherein
one of the two electrodes is located outside the container during the determination of the electrical parameter, and the other electrode is either located outside the container in such a way that the container is arranged between the two electrodes, or the other electrode is located at least in part inside the container in such a way that at least one wall portion of the container is located between the electrodes.

3. The method according to claim 1,
wherein
the container is made of a pulp comprised of paper or cardboard.

4. The method according to claim 1,
wherein
an alternating electric field is built up between the electrodes.

5. The method according to claim 1,
wherein
the measurement is carried out at at least two different frequencies.

6. The method according to claim 1,
wherein
the measurement of the characteristic parameter takes place during a movement of the container.

7. The method according to claim 1,
wherein
the parameter which is characteristic for the container is a moisture value and/or a wall thickness of at least one wall portion of the container.

8. The method according to claim 1,
wherein
the electrical parameter is an amplitude and/or a phase of the electric field.

9. The method according to claim 1,
wherein
a wall thickness is determined when the residual moisture is known and/or constant.

10. The method according to claim 1,
wherein
the at least one characteristic parameter of the container is determined sectionally in the longitudinal direction of the container, and/or the at least one characteristic parameter of the container is determined depending on the longitudinal direction of the container.

11. The method according to claim 1,
wherein
the container is rotated during the measurement.

12. An apparatus for determining container properties, having a transport device which transports a container to be examined along a predetermined transport path, wherein this container has a bottom region, a main body which serves to receive a filling material, and a mouth region, and wherein this container is made at least in part of a material containing paper or cardboard,
wherein
the apparatus has at least two electrodes which are arranged such that at least one wall of the container is temporarily arranged between these two electrodes, wherein an electrical voltage can be applied to the electrodes, and a measuring device is provided which is configured to determine at least one electrical parameter which is characteristic of an electrical field which is built up between the electrodes, and furthermore an evaluation device is provided which is configured to determine at least one parameter characteristic of the container from this electrical parameter.

13. The apparatus according to claim 12,
wherein
at least one electrode has a plurality of separately activatable electrode portions.

14. The apparatus according to claim 12,
wherein
the apparatus comprises a rotating device which is configured to rotate the containers with respect to their longitudinal direction.

15. An arrangement for processing containers, having an apparatus according to claim 14, and having a drying device configured for drying the containers, wherein the drying device is controllable depending on the characteristic parameter of the container.
